(19) United States
(12) Patent Application Publication
(10) Pub. No.: US 2023/0346867 A1
(43) Pub. Date: Nov. 2, 2023

(54) WELLNESS SPRAY FORMULATIONS

(71) Applicant: WIPARO BIOMED LIMITED, GALWAY (IE)

(72) Inventor: David SCHMIDT, Winter Park, FL (US)

(73) Assignee: WIPARO BIOMED LIMITED, GALWAY (IE)

(21) Appl. No.: 18/310,663

(22) Filed: May 2, 2023

Related U.S. Application Data
(60) Provisional application No. 63/337,583, filed on May 2, 2022.

(51) Int. Cl. A61K 36/63; A61K 9/00; A61K 36/15; A61K 36/185; A61K 36/47; A61K 36/53; A61K 36/537

(52) U.S. Cl. CPC A61K 36/63; A61K 9/0073; A61K 36/15; A61K 36/185; A61K 36/47; A61K 36/53; A61K 36/537

(57) ABSTRACT

Embodiments include a well spray used when first awakening, and then again before exercise. Embodiments produce a very noticeable increase in strength and stamina in the gym. Another embodiment includes an evening spray that helps a user feel sleepy after about 10 minutes, wherein the quality of sleep is longer and deeper.

This patent application claims the benefit of U.S. Provisional Patent Application Ser. No. 63/337,583, filed 2 May 2022, the specification of which is hereby incorporated herein by reference.

### BACKGROUND OF THE INVENTION

### Field of the Invention

One or more embodiments of the invention are related to the field of aerosol compositions. More particularly, but not by way of limitation, one or more embodiments of the invention enable one or more wellness sprays that improve overall energy, vitality, wellness index, sleep quality, bioelectric flow in meridians and bioelectrical energy of organs.

### Description of the Related Art

Aromatherapy is one method in which compositions are utilized for personal care and general well-being. Aromatherapy requires use of a diffuser to disperse natural oils, generally extracted from plants to enhance psychological and physical well-being. Known sprays are generally directed towards helping with unwanted odors in gyms and masking foul odors in cars, etc. There are no known sprays that produce a very noticeable increase in strength and stamina in the gym or help a user to feel sleepy or otherwise improve the quality of sleep shortly after use.

For at least the limitations described above there is a need for a wellness spray that increases strength and stamina and another spray that helps a user feel sleepy shortly after application, wherein the quality of sleep is longer and deeper.

### BRIEF SUMMARY OF THE INVENTION

One or more embodiments described in the specification are related to a wellness spray that may be used daily or as a pre-workout product, helping to improve and increase a user's strength and stamina, and another spray that helps a user to balance their hormones, especially improving their quality of sleep shortly after application, wherein the quality of sleep is longer and deeper.

### DETAILED DESCRIPTION OF THE INVENTION

One embodiment of the invention is a wellness spray that helps to support increases in strength and stamina. This product may be applied in the morning, and optionally may also be applied prior to exercise as a pre-workout product. A second embodiment of the invention is a spray that helps a user improve the quality of their sleep, thus improving and supporting natural hormone production. The product has also demonstrated the ability to help an individual enter a state of relaxation and sleep shortly after application, wherein the quality of sleep is longer and deeper.

In the following exemplary description, numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

On formulation that increases strength and stamina is composed of the following ingredients:

Dry Components:
3 grams Canadian Pine Pollen
3 grams Tongkat Ali
1 gram Polyrhachis Extract

Oil Components:
10 drops Pine Oil
10 drops Blue Spruce Oil
10 drops Ginger Oil
10 drops Sage Oil

One or more embodiments of the formulation that increases strength and stamina is composed of the following ingredients in the listed ranges:

Dry Components:
0.5-5 grams Canadian Pine Pollen
0.5-5 grams Tongkat Ali
0.5-5 grams Polyrhachis Extract

Oil Components:
1-20 drops Pine Oil
1-20 drops Blue Spruce Oil
1-20 drops Ginger Oil
1-20 drops Sage Oil

The above dry herbal ingredients are placed into a glass bottle along with 300 ml 153 proof (76.5%) grain alcohol. This glass bottle is then placed into a vibrational mixer and blended for 10 minutes. The bottle is removed from the mixer, and the liquid is filtered to remove the heavy solids.

To this liquid herbal extract is added all of the essential oils listed above. The glass bottle is returned to the vibrational mixer and blended for 5 minutes.

At this point the mixture may be added to a glass bottle with a spray top and is now ready for use.

To apply, the liquid product is sprayed to the wrist and then the scent is inhaled 7 or 8 times. This product will promote feelings of energy and overall wellness.

Embodiments of the invention may be utilized upon awakening and then before a workout.

On formulation that aids in sleep is composed of the following ingredients:

Dry Components:
3 grams Canadian Pine Pollen
3 grams Tongkat Ali
6.4 grams Mucuna Pruriens

Oil Components:
10 drops Pine Oil
10 drops Jasmine Oil
10 drops Lavendar Oil
10 drops Clary Sage Oil

One or more embodiments of the formulation that aids in sleep is composed of the following ingredients in the listed ranges:

Dry Components:
0.5-5 grams Canadian Pine Pollen
0.5-5 grams Tongkat Ali
0.5-12 grams Mucuna Pruriensåπ

Oil Components: oeoe
1-20 drops Pine Oil
1-20 drops Jasmine Oil
1-20 drops Lavendar Oil
1-20 drops Clary Sage Oil

The above dry herbal ingredients are placed into a glass bottle along with 300 ml 153 proof (76.5%) grain alcohol. This glass bottle is then placed into a vibrational mixer and blended for 10 minutes. The bottle is removed from the mixer, and the liquid is filtered to remove the heavy solids.

To this liquid herbal extract is added all of the essential oils listed above. The glass bottle is returned to the vibrational mixer and blended for 5 minutes.

At this point the mixture may be added to a glass bottle with a spray top and is now ready for use.

To apply, the liquid product is sprayed to the wrist and then the scent is inhaled 7 or 8 times. This product will promote feelings of rest and relaxation.

While the invention herein disclosed has been described by means of specific embodiments and applications thereof, numerous modifications and variations could be made thereto by those skilled in the art without departing from the scope of the invention set forth in the claims.

What is claimed is:

1. A wellness spray comprising:
Dry Components:
0.5-5 grams Canadian Pine Pollen
0.5-5 grams Tongkat Ali
0.5-12 grams Mucuna Pruriens
or
0.5-5 grams Polyrhachis Extract; and,
Oil Components:
1-20 drops Pine Oil;
1-20 drops Jasmine Oil;
1-20 drops Lavendar Oil;
1-20 drops Clary Sage Oil.

2. A wellness spray comprising:
Dry Components:
3 grams Canadian Pine Pollen;
3 grams Tongkat Ali;
1 gram Polyrhachis Extract
or
6.4 grams Mucuna Pruriens; and,
Oil Components:
10 drops Pine Oil;
10 drops Jasmine Oil;
10 drops Lavendar Oil;
10 drops Clary Sage Oil.
